SEQUENCE LISTING (9 sequences)

SEQ ID NO: 1 - protein, 112 aa, Artificial
  Synthetic construct
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 154 aa, Artificial
  Synthetic construct
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 54 aa, Artificial
  Synthetic construct
MNSDSECPLSHDGYCLHDGVCMYIEALDKYACNCVVGYIGERCQYRDLKWWELR

SEQ ID NO: 4 - protein, 109 aa, Artificial
  Synthetic construct
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 5 - protein, 565 aa, Artificial
  Synthetic construct
DWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGA

SEQ ID NO: 6 - protein, 6 aa, Artificial
  Synthetic construct
GGGGGG

SEQ ID NO: 7 - protein, 5 aa, Artificial
  Synthetic construct
GGGGT

SEQ ID NO: 8 - protein, 6 aa, Artificial
  Synthetic construct
GGGPPP

SEQ ID NO: 9 - protein, 7 aa, Artificial
  Synthetic construct
GGGAPPP

(19) United States
(12) Patent Application Publication
(10) Pub. No.: US 2023/0147855 A1
(43) Pub. Date: May 11, 2023

(54) METHODS OF GENERATING PLURIPOTENT STEM CELL-DERIVED VASCULAR SMOOTH MUSCLE CELLS, USES, AND COMPOSITION RELATED THERETO

(71) Applicant: EMORY UNIVERSITY, Atlanta, GA (US)

(72) Inventors: Young-Sup YOON, Atlanta, GA (US); Kyung Hee KIM, Atlanta, GA (US)

(21) Appl. No.: 17/917,315

(22) PCT Filed: Apr. 9, 2021
(86) PCT No.: PCT/US2021/026659

§ 371 Date: Oct. 6, 2022

Related U.S. Application Data
(60) Provisional application No. 63/007,698, filed on Apr. 9, 2020.

(51) Int. Cl. C12N 5/077; A61K 35/34

(52) U.S. Cl. CPC C12N 5/0661; A61K 35/34; C12N 2501/115; C12N 2501/15; C12N 2501/11; C12N 2501/135; C12N 2509/00; C12N 2506/45

(57) ABSTRACT

This disclosure relates to methods of making vascular smooth muscle like cells from precursor stem cells. In certain embodiments the vascular smooth muscle like cells are able to contract in response to vasoactive agents, In certain embodiments, the methods comprise contacting pluripotent stem cells with a mesoderm induction growth medium, followed by replicating the cells in a serum-free vascular smooth muscle cell growth medium in the presence of collagen, and purifying replicated cells that express cadherin-2. In certain embodiments, the purified cells are used to treat or prevent a cardiovascular disease or condition.

### CROSS-REFERENCE TO RELATED APPLICATIONS

This application claims the benefit of U.S. Provisional Application No. 63/007,698 filed Apr. 9, 2020. The entirety of this application is hereby incorporated by reference for all purposes.

### STATEMENT REGARDING FEDERALLY SPONSORED RESEARCH OR DEVELOPMENT

This invention was made with government support under HL127759 and DK108245 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 19072PCT_ST25.txt. The text file is 10 KB, was created on Apr. 8, 2021, and is being submitted electronically via EFS-Web.

### BACKGROUND

Dysfunctional blood vessels typically result after cardiovascular ischemic conditions such as myocardial infarction (MI) and peripheral arterial disease (PAD). These diseases often involve atherosclerotic occlusion of blood vessels, leading to low blood circulation and eventual tissue necrosis. Thus, there is a need to identify improved methods of managing the results of vascular dysfunction.

In addition to endothelial cells, vascular smooth muscle cells (VSMCs) are present in blood vessels. VSMCs exist in a contractile (differentiated) phenotype characterized by expression of smoothelin (SMTN) and smooth muscle myosin heavy chain. Loss of contractility and acquisition of an epithelial phenotype of vascular smooth muscle cells (VSMCs) are implicated in proliferative vascular pathologies such as vascular inflammation, plaque formation, atherosclerosis, restenosis, and pulmonary hypertension. However, when isolated and cultured in the presence of serum, VSMCs transform to a less differentiated state referred to as a synthetic phenotype that proliferative. Thus, there is a need to identify improved methods of generating and maintaining VSMCs to maintain a contractile phenotype.

Cheung et al. report generating human vascular smooth muscle subtypes. Nat Biotechnol. 2012, 30(2): 165-173. Patsch et al. report the generation of vascular endothelial and smooth muscle cells from human pluripotent stem cells. Nat Cell Biol 2015, 17:994-1003.

References cited herein are not an admission of prior art.

### SUMMARY

This disclosure relates to methods of making vascular smooth muscle like cells from precursor stem cells. In certain embodiments the vascular smooth muscle like cells are able to contract in response to vasoactive agents. In certain embodiments, the methods comprise contacting pluripotent stem cells with a mesoderm induction growth medium, followed by replicating the cells in a serum-free vascular smooth muscle cell growth medium in the presence of collagen, and purifying replicated cells that express cadherin-2. In certain embodiments, the purified cells are used to treat or prevent a cardiovascular disease or condition.

In certain embodiments, this disclosure relates to methods of producing vascular smooth muscle like cells in a contractile phenotype comprising transforming pluripotent stem cells into cells that express smoothelin and smooth muscle myosin heavy chain and purifying cells that express cadherin-2 providing a purified composition of cadherin-2 expressing vascular smooth muscle like cells in a contractile phenotype. In certain embodiments, the method further comprises replicating cadherin-2 expressing vascular smooth muscle like cells.

In certain embodiments, this disclosure relates to serum-free methods making vascular smooth muscle like cells comprising, a) contacting pluripotent stem cells with a mesoderm induction growth medium for a day or more, wherein the mesoderm induction growth medium comprises: 1) rho-associated protein kinase inhibitor, 2) glycogen synthase kinase-3 inhibitor, and 3) basic fibroblast growth factor; under conditions such that the pluripotent stem cells form induced mesodermal-like cells; contacting the induced mesodermal-like cells with a first vascular smooth muscle cell growth medium for a day or more, wherein the first vascular smooth muscle cell growth medium comprises: 1) transforming growth factor-beta, 2) epidermal growth factor, and 3) platelet-derived growth factor; under conditions such that the mesodermal-like cells form induced vascular smooth muscle like cells; c) contacting the induced vascular smooth muscle like cells with a protease and/or collagenase under conditions such that induced vascular smooth muscle like cells detach from each other providing detached induced vascular smooth muscle like cells; d) replicating the detached induced vascular smooth muscle like cells by exposure to collagen and the first vascular smooth muscle cell growth medium for a day or more providing replicated vascular smooth muscle like cells; e) contacting replicated vascular smooth muscle like cells with a second vascular smooth muscle cell growth medium for a day or more, wherein the second vascular smooth muscle cell growth medium comprises: 1) transforming growth factor-beta, 2) epidermal growth factor, and 3) platelet-derived growth factor; under conditions such that the replicated vascular smooth muscle like cells form a second batch of induced vascular smooth muscle like cells; and f) purifying the second batch of induced vascular smooth muscle like cells by selecting cells that express cadherin-2, providing purified cadherin-2 expressing induced vascular smooth muscle like cells.

In certain embodiments, the concentration of transforming growth factor-beta in the second vascular smooth muscle cell growth medium is increased compared to the concentration of transforming growth factor-beta in the first vascular smooth muscle cell growth medium.

In certain embodiments, the concentration of platelet-derived growth factor in the second vascular smooth muscle cell growth medium is decreased compared to the concentration of platelet-derived growth factor in the first vascular smooth muscle cell growth medium.

In certain embodiments, the rho-associated protein kinase inhibitor is trans-4-[(1R)-1-aminoethyl]-N-4-pyridinylcyclohexanecarboxamide (Y-27632) or salt thereof.

In certain embodiments, the glycogen synthase kinase-3 inhibitor is 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR-99021) or salt thereof.

In certain embodiments, the pluripotent stem cells are embryonic stem (ES) cells or induced pluripotent stem (iPS) cells.

In certain embodiments, said contacting pluripotent stem cells with a mesoderm induction growth medium for a day or more comprises contacting for four days.

In certain embodiments, said contacting pluripotent stem cells with a mesoderm induction growth medium for a day or more is for not more than five days.

In certain embodiments, said contacting the induced mesodermal-like cells with a first vascular smooth muscle cell growth medium for a day or more comprises contacting for twenty days.

In certain embodiments, said contacting the induced mesodermal-like cells with a first vascular smooth muscle cell growth medium for a day or more, is for not more than 21 days.

In certain embodiments, said replicating the detached induced vascular smooth muscle like cells by exposure to collagen and the first vascular smooth muscle cell growth medium for a day or more comprises replicating for fifteen days.

In certain embodiments, said replicating the detached induced vascular smooth muscle like cells by exposure to collagen and the first vascular smooth muscle cell growth medium for a day or more is for not more than 16 days.

In certain embodiments, said contacting replicated vascular smooth muscle like cells with a second vascular smooth muscle cell growth medium for a day or more comprises contacting for twenty-five days or more.

In certain embodiments, said contacting replicated vascular smooth muscle like cells with a second vascular smooth muscle cell growth medium for a day or more is not more than 26 days.

In certain embodiments, the methods disclosed herein further comprises the step of replicating the purified cadherin-2 expressing induced vascular smooth muscle like cells by exposing the purified cadherin-2 expressing induced vascular smooth muscle like cells to collagen and the second vascular smooth muscle cell growth medium for a day or more.

In certain embodiments, said selecting cells that express cadherin-2 comprises contacting the cells with an anti-cadherin-2 antibody, marking the antibody with a fluorescent antibody, and selecting cells by fluorescence-activated cell sorting and selecting cells by fluorescence-activated cell sorting.

In certain embodiments, this disclosure relates to compositions and growth medium comprising cells made by methods disclosed herein.

In certain embodiments, this disclosure relates to methods of treating or preventing a cardiovascular disease or condition comprising administering an effective amount of cells made by methods disclosed herein to a subject in need thereof. In certain embodiments, the pluripotent stem cells are induced pluripotent stem cells derived from the subject. In certain embodiments, the subject is diagnosed with myocardial infarction, vascular inflammation, plaque formation, atherosclerosis, restenosis, and pulmonary hypertension.

In certain embodiments, vascular smooth muscle like cells contract in response to vasoactive agents such as carbachol, endothelin-1 (ET-1) or KCl.

In certain embodiments, vascular smooth muscle like cells are mixed with or administered in combination with and endothelial cells or endothelial like cells resulting in improved blood flow restoration compared with the administration of endothelial cells or endothelial like cells alone.

In certain embodiments, vascular smooth muscle like cells are mixed with or administered in combination with and endothelial cells or endothelial like cells creating capillary-like tubes.

### BRIEF DESCRIPTION OF THE SEVERAL VIEWS OF THE DRAWINGS

FIG. 1 illustrates a process to generate VSMCs from hiPSCs.

FIG. 2 shows data on gene expression patterns of hiPSC-VSMCs during differentiation.

FIG. 3 shows a comparison of gene expression pattern of hiPSC-VSMCs with hAoSMCs.

FIG. 4 shows data indicating reduced pluripotency-related gene expression in CDH2-positive hiPSC-VSMCs at day 57.

FIG. 5 shows the percentage of hiPSC-VSMCs positive for VSMC-specific markers, day 63.

FIG. 6A shows data indicating increased intracellular calcium flux of hiPSC-VSMCs.

FIG. 6B shows data indicating increased contractility of hiPSC-VSMCs.

FIG. 7 shows quantification of tube formation of hiPSC-VSMC with HUVEC.

FIG. 8 shows data on MMP2 and MMP9 gene expression in hiPSC-VSMCs.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

“Subject” means any animal, but is preferably a mammal, such as, for example, a human, monkey, mouse, or rabbit.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

The terms “smooth muscle α-actin” and “aortic smooth muscle actin” refer to the gene products of ACTA2 on chromosome 10. Homo sapiens actin alpha 2, smooth muscle (ACTA2), transcript variant 1, mRNA has NCBI Reference Sequence: NM_001141945.2.

The terms “smooth muscle myosin heavy chain” and “SMHC” refer to the gene products of MYH11 on Homo sapiens chromosome 16. Homo sapiens myosin heavy chain 11 (MYH11), transcript variant SM2B, mRNA has NCBI Reference Sequence: NM_001040113.2.

The terms “transgelin” and “SM22-alpha” refer to the gene products of TAGLN on Homo sapiens (human) chromosome 11. Homo sapiens transgelin (TAGLN), transcript variant 2, mRNA has NCBI Reference Sequence: NM_003186.5.

The terms “calponin 1” and “CNN1” refer to the gene products of CNN1 on Homo sapiens (human) chromosome 19. Homo sapiens calponin 1 (CNN1), transcript variant 1, mRNA has NCBI Reference Sequence: NM_001299.6.

The terms, “caldesmon 1” and “CALD1” refer to the gene products of CALD1 on Homo sapiens chromosome 7. Homo sapiens caldesmon 1 (CALD1), transcript variant 2, mRNA has NCBI Reference Sequence: NM_004342.7.

The terms “smoothelin” and “SMTN” refer to the gene products of SMTN on Homo sapiens chromosome 22. Homo sapiens smoothelin (SMTN), transcript variant 4, mRNA has NCBI Reference Sequence: NM_001207017.1.

Glycogen synthase kinase-3 (GSK-3) is a serine/threonine kinase. It transfers a phosphate group to either the serine or threonine residues of its substrates. GSK-3 phosphorylation modulates biological processes, including metabolism (glucose regulation), cell signaling, cellular transport, apoptosis, and proliferation. A “GSK-3 inhibitor” refers to a molecule that interferes with substrate phosphorylation. In certain embodiments, a GSK-3 inhibitor contemplated herein is selected from: 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR99021); N-6-[2-[[4-(2,4-dichlorophenyl)-5-(1H-imidazol-1-yl)-2-pyrimidinyl]amino]ethyl]-3-nitro-2,6-pyridinediamine (CHIR-98014); 3-(1,3-dihydro-3-oxo-2H-indol-2-ylidene)-1,3-dihydro-2H-indol-2-one (Indirubin); (2′Z,3′E)-6-bromoindirubin-3′-oxime (BIO); (2′Z,3′E)-6-bromoindirubin-3′-acetoxime (BIO-acetoxime); 3-(2,4-dichlorophenyl)-4-(1-methyl-1H-indol-3-yl)-1H-pyrrole-2,5-dione (SB216763); 3-[6-(3-Aminophenyl)-7H-pyrrolo[2,3-d]pyrimidin-4-yloxy]phenol (TWS119); 4-benzyl-2-(naphthalen-1-yl)-[1,2,4]thiadiazolidine-3,5-dione (Tideglusib); 3-[(3-chloro-4-hydroxyphenyl)amino]-4-(2-nitrophenyl)-1H-pyrrol-2,5-dione (SB415286); 3-amino-6-[4-[(4-methyl-1-piperazinyl)sulfonyl]phenyl]-N-3-pyridinyl-2-pyrazinecarboxamide (AZD2858); 2-hydroxy-3-[5-[(morpholin-4-yl)methyl]pyridin-2-yl]-1H-indole-5-carbonitrile (AZD1080); N-(4-methoxybenzyl)-N′-(5-nitro-1,3-thiazol-2-yl)urea (AR-A014418); 3-[9-fluoro-1,2,3,4-tetrahydro-2-(1-piperidinylcarbonyl)pyrrolo[3,2,1-jk][1,4]benzodiazepin-7-yl]-4-imidazo[1,2-a]pyridin-3-yl-1h-pyrrole-2,5-dione (LY2090314); and 3-(4-fluorophenylethylamino)-1-methyl-4-(2-methyl-1H-indol-3-yl)-1H-pyrrole-2,5-dione (IM-12) or salts thereof.

Rho-associated protein kinase (ROCK) is a serine-threonine kinase and plays a role in cellular phenomena including mediating vasoconstriction and vascular remodelling. ROCK is a downstream effector protein of the small GTPase Rho. A “ROCK inhibitor” refers to a molecule that interferes with substrate phosphorylation. In certain embodiments, a ROCK inhibitor contemplated herein is selected fasudil; ripasudil; netarsudil; N-[(3-hydroxyphenyl)methyl]-N′-[4-(4-pyridinyl)-2-thiazolyl]urea (RKI-1447); trans-4-[(1R)-1-aminoethyl]-N-4-pyridinylcyclohexanecarboxamide (Y-27632); 4-[4-(trifluoromethyl)phenyl]-N-(6-fluoro-1H-indazol-5-yl)-2-methyl-6-oxo-1,4,5,6-tetrahydro-3-pyridinecarboxamide (GSK-429286); and 4-(1-aminoethyl)-N-(1H-pyrrolo(2,3-b)pyridin-4-yl)cyclohexanecarboxamide (Y-30141) or salts thereof.

The terms, “transforming growth factor β,” TGF-β,” and “TGF-beta” refer to a cytokine that is secreted by various cell types and regulates homeostasis in normal epithelial cells. There are three TGF-β isoforms. TGF-beta isoform 1 is most prominent. Human recombinant TGF-β is commercially available as a 25.0 kDa protein linked by a single disulfide bond with each subunit containing 112 aa having the following sequence:

(SEQ ID NO: 1)
ALDTNYCFSSTEKNCCVRQLYIDFRKDLGWKWIHE
PKGYHANFCLGPCPYIWSLDTQYSKVLALYNQHNP
GASAAPCCVPQALEPLPIVYYVGRKPKVEQLSNMI
VRSCKCS.

The terms, “basic fibroblast growth factor” or “bFGF” refer to a protein that has the β-trefoil structure which binds to FGF receptor (FGFR) family members. Human recombinant bFGF is commercially available in the form of a 154 amino acid protein having the following sequence:

(SEQ ID NO: 2)
AAGSITTLPALPEDGGSGAFPPGHFKDPKRLYCKN
GGFFLRIHPDGRVDGVREKSDPHIKLQLQAEERGV
VSIKGVCANRYLAMKEDGRLLASKCVTDECFFFER
LESNNYNTYRSRKYTSWYVALKRTGQYKLGSKTGP
GQKAILFLPMSAKS.

The terms, “Epidermal growth factor” and “EGF” refer to a protein which is about a 6-kDa. Human EGF gene encodes preproprotein that is proteolytically processed to generate a peptide that functions to stimulate the division of epidermal and other cells. Human recombinant EGF is commercially available in the form of a 54 amino acid protein having the following sequence:

(SEQ ID NO: 3)
MNSDSECPLSHDGYCLHDGVCMYIEALDKYACNCV
VGYIGERCQYRDLKWWELR.

Platelet-Derived Growth Factors (PDGFs) are disulfide-linked dimers consisting of two polypeptide chains, designated PDGF-A and PDGF-B chains. The three naturally occurring PDGFs are PDGF-AA, PDGF-BB and PDGF-AB. Human recombinant PDGF-BB is commercially available as a 24.3 kDa disulfide-linked homodimer of two β chains (218 total amino acids) having the following sequence:

(SEQ ID NO: 4)
SLGSLTIAEPAMIAECKTRTEVFEISRRLIDRTNA
NFLVWPPCVEVQRCSGCCNNRNVQCRPTQVQLRPV
QVRKIEIVRKKPIFKKATVTLEDHLACKCETVAAA
RPVT.

Cadherin-2 (CDH2), also known as N-cadherin and CD325 is a transmembrane, homophilic glycoprotein which belongs to the calcium-dependent cell adhesion molecule family. Human recombinant CDH2 is commercially available as a polypeptide chain having the following sequence:

(SEQ ID NO: 5)
DWVIPPINLPENSRGPFPQELVRIRSDRDKNLSLR
YSVTGPGADQPPTGIFIINPISGQLSVTKPLDREQ
IARFHLRAHAVDINGNQVENPIDIVINVIDMNDNR
PEFLHQVWNGTVPEGSKPGTYVMTVTAIDADDPNA
LNGMLRYRIVSQAPSTPSPNMFTINNETGDIITVA
AGLDREKVQQYTLIIQATDMEGNPTYGLSNTATAV
ITVTDVNDNPPEFTAMTFYGEVPENRVDIIVANLT
VTDKDQPHTPAWNAVYRISGGDPTGRFAIQTDPNS
NDGLVTVVKPIDFETNRMFVLTVAAENQVPLAKGI
QHPPQSTATVSVTVIDVNENPYFAPNPKIIRQEEG
LHAGTMLTTFTAQDPDRYMQQNIRYTKLSDPANWL
KIDPVNGQITTIAVLDRESPNVKNNIYNATFLASD
NGIPPMSGTGTLQIYLLDINDNAPQVLPQEAETCE
TPDPNSINITALDYDIDPNAGPFAFDLPLSPVTIK
RNWTITRLNGDFAQLNLKIKFLEAGIYEVPIIITD
SGNPPKSNISILRVKVCQCDSNGDCTDVDRIVGAG
LGTGA.

As used herein the term “allogeneic” refers to cells that are genetically dissimilar because they are not derived from the same person. Cells derived from the same person are designated as “syngeneic.”

Embryonic stem cells (ESCs) originate from the inner cell mass of mammalian blastocysts which occur 5-7 days after fertilization. ESCs remain undifferentiated indefinitely under defined conditions and differentiate into so-called embryonic bodies when cultivated in vitro. Having pluripotency, they are capable of differentiating into all cell types. Adult stem cells (somatic cells), such as hematopoietic, neural, and mesenchymal stem cells have an ability to become more than one cell type but do not have the ability to become any cell type.

Induced pluripotent stem cells (iPSCs) are differentiated cells reprogrammed to return to a pluripotent stage. Reprogrammed fully differentiated cells may be accomplished using genes involved in the maintenance of ESC pluripotency, e.g., Oct3/4, Sox2, c-Myc, Klf4, and combinations thereof. The term “induced pluripotent stem cells” refers to cells that are reprogrammed from somatic or adult stems cells to an embryonic stem cell (ESC)-like pluripotent state. See Takahashi et al. “Induction of pluripotent stem cells from mouse embryonic and adult fibroblast cultures by defined factors,” Cell, 2006, 126(4):663-676. Park et al. report reprogramming of human somatic cells to pluripotency with defined factors, Nature, 2008, 451(7175):141-146. Thus, making iPSCs in cells can typically be accomplished by in trans expression of OCT4, SOX2, KLF4 and c-MYC. Colonies appear and resemble ESCs morphologically. Alternatively, certain multipotent stem cells may require less than all of the four transcripts, e.g., cord blood CD133+ cells require only OCT4 and SOX2 to generate iPSCs. For additional guidance in generating iPSCs, see Gonzalez et al. “Methods of making induced pluripotent stem cells: reprogramming a la carte,” Nature Reviews Genetics, 2011, 12:231-242.

Induced pluripotent stem cells typically express alkaline phosphatase, Oct 4, Sox2, Nanog, and/or other pluripotency-promoting factors. It is not intended that induced pluripotent stem cells be entirely identical to embryonic cells. Induced pluripotent stem cells may not necessarily be capable of differentiating into any type of cell. TRA-1-60, TRA-1-8, or a combination thereof may be used to identify human iPSCs.

In certain embodiments, this disclosure contemplates that induced pluripotent stem cells are derived from adult stem cells or mesenchymal stem cells. These terms include the cultured (self-renewed) progeny of cell populations. The term “mesenchymal stromal cells” or “mesenchymal stem cells” refers to the subpopulation of fibroblast or fibroblast-like nonhematopoietic cells with properties of plastic adherence and capable of in vitro differentiation into cells of mesodermal origin which may be derived from bone marrow, adipose tissue, Wharton's jelly in umbilical cord, umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells.

In certain embodiments, this disclosure contemplates that induced pluripotent stem cells are derived from human adipose stem cells. Sun et al. Proc Natl Acad Sci USA., 2009, 106(37):15720-15725 report induced pluripotent stem (iPS) cells can be generated from adult human adipose stem cells (hASCs) freshly isolated from patients.

In certain embodiments, this disclosure contemplates that induced pluripotent stem cells are derived bone marrow derived mesenchymal stromal cells. Bone marrow derived mesenchymal stromal cells are typically expanded ex vivo from bone marrow aspirates to confluence. Certain mesenchymal stromal/stem cells (MSCs) share a similar set of core markers and properties. Certain mesenchymal stromal/stem cells (MSCs) may be defined as positive for CD105, CD73, and CD90 and negative for CD45, CD34, CD14 or CD11b, CD79α or CD19, and HLA-DR surface markers, and have the ability to adhere to plastic. See Dominici et al. “Minimal criteria for defining multipotent mesenchymal stromal cells,” The International Society for Cellular Therapy position statement, Cytotherapy, 2006, 8(4):315-317.

As used herein, the terms “growth medium” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Additionally, growth medium may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganese chloride, ethanolamine, and sodium pyruvate. Other contemplated components in the growth medium include ascorbic acid, L-alanine, zinc sulfate, human transferrin, albumin, and insulin.

Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8). Various growth mediums are known in the art.

Dulbecco's modified Eagle's medium (DMEM) is a growth medium which contains additional components such as glycine, serine and ferric nitrate with increased amounts of vitamins, amino acids, and glucose as indicated in Table 1 below.

TABLE 1
Composition of Dulbecco's modified Eagle's medium

| Components | Concentration (mg/L) |
|---|---|
| Amino Acids | |
| Glycine | 30.0 |
| L-Arginine hydrochloride | 84.0 |
| L-Cystine 2HCl | 63.0 |
| L-Glutamine | 584.0 |
| L-Histidine hydrochloride-H₂O | 42.0 |
| L-Isoleucine | 105.0 |
| L-Leucine | 105.0 |
| L-Lysine hydrochloride | 146.0 |
| L-Methionine | 30.0 |
| L-Phenylalanine | 66.0 |
| L-Serine | 42.0 |
| L-Threonine | 95.0 |
| L-Tryptophan | 16.0 |
| L-Tyrosine disodium salt dihydrate | 104.0 |
| L-Valine | 94.0 |
| Vitamins | |
| Choline chloride | 4.0 |
| D-Calcium pantothenate | 4.0 |
| Folic Acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Inorganic Salts | |
| Calcium Chloride (CaCl₂) (anhyd.) | 200.0 |
| Ferric Nitrate (Fe(NO₃)₃:9H₂O) | 0.1 |
| Magnesium Sulfate (MgSO₄) (anhyd.) | 97.67 |
| Potassium Chloride (KCl) | 400.0 |
| Sodium Bicarbonate (NaHCO₃) | 3700.0 |
| Sodium Chloride (NaCl) | 6400.0 |
| Sodium Phosphate monobasic (NaH₂PO₄—H₂O) | 125.0 |
| Other Components | |
| Phenol Red | 15.0 |

Ham's F-12 medium has high levels of amino acids, vitamins, and other trace elements. Putrescine and linoleic acid are included in the formulation. See Table 2 below.

TABLE 2
Composition of the Ham's F-12 medium

| Substance | Concentration (mg/L) |
|---|---|
| NaCI | 7599 |
| KCI | 223.6 |
| Na₂HPO₄ | 142 |
| CaCl₂•2H₂O | 44 |
| MgCl₂ | 122 |
| FeSO₄•7H₂O | 0.834 |
| CuSO₄•5H₂O | 0.00249 |
| ZnSO₄•7H₂O | 0.863 |
| D-glucose | 1802 |
| Na-pyruvate | 110 |
| Phenol red | 1.2 |
| NaHCO3 | 1176 |
| L-alanine | 9 |
| L-arginine•HCl | 211 |
| L-asparagine 13.2 | 13.2 |
| L-aspartic acid | 13.3 |
| L-cysteine•HCI | 31.5 |
| L-glutamine | 146 |
| L-glutamic acid | 14.7 |
| Glycine | 7.5 |
| L-histidine•HCI•H₂O | 21 |
| L-isoleucine | 4 |
| L-leucine | 13 |
| L-lysine•HCI | 36.5 |
| L-methionine | 4.47 |
| L-phenylalanine | 5 |
| L-proline | 34.5 |
| L-serine | 10.5 |
| L-threonine | 12 |
| L-tryptophan | 2 |
| L-tyrosine | 5.4 |
| L-valine | 11.7 |
| Biotin | 0.0073 |
| D-Ca-pantothenate | 0.48 |
| Choline chloride | 14 |
| Folic acid | 1.3 |
| i-inositol | 18 |
| Nicotinic acid amid | 0.037 |
| Pyridoxin•HCI | 0.062 |
| Riboflavin | 0.038 |
| Thiamine•HCI | 0.34 |
| Vitamin B12 | 1.36 |
| Hypoxanthine | L 4.1 |
| Thymidine | 0.73 |
| Lipoic acid | 0.21 |
| Linoleic acid | 0.084 |
| Putrescine•2HCI | 0.161 |

In certain embodiments, the disclosure contemplates a growth medium disclosed herein using a DMEM/F-12 medium which is a mixture of DMEM and Ham's F-12. In certain embodiments, the growth medium may contain antimicrobial agents or combinations of antimicrobial agents, e.g., antimycotic which contains the antibiotic penicillin, streptomycin, and the antifungal agent amphotericin B.

Human embryonic stem cells and induced pluripotent stem cells can be cultured in the presence of basic fibroblast growth factor (bFGF), e.g., on fibroblast feeder layers or in unconditioned medium (UM) supplemented with more than 100 ng/mL bFGF. A growth medium designated (TeSR1™) is composed of a DMEM/F12 base supplemented with human serum albumin, vitamins, antioxidants, trace minerals, specific lipids and cloned growth factors. TeSR medium has 18 components added to a DMEM/F12 base medium that itself has 52 components. A list of the individual components in TeSR1™ are provide below as reported with associated concentration in the supplemental materials of Ludwig et al., Derivation of human embryonic stem cells in defined conditions, Nature Biotechnology volume 24, pages185-187 (2006).

Inorganic salts
Calcium chloride (Anhydrous)
HEPES
Lithium chloride (LiCl)
Magnesium chloride (Anhydrous)
Magnesium sulfate (MgSO₄)
Potassium chloride (KCl)
Sodium bicarbonate (NaHCO₃)
Sodium chloride (NaCl)
Sodium phosphate, dibasic (Anhydrous)
Sodium phosphate, mono. (NaH₂PO₄—H₂0)
Trace minerals
Ferric Nitrate (Fe(NO₃)₃—9H₂O)
Ferric sulfate (FeSO₄—7H₂O)
Cupric sulfate (CuSO₄—5H₂O)
Zinc sulfate (ZnSO₄—7H₂O)
Ammonium metavanadate NH₄VO₃
Mangenous sulfate monohydrate MnSO₄—H₂O
NiSO₄ 6H₂O
Selenium
Sodium metasilicate Na₂SiO₃ 9H₂O
SnCl₂
Molybdic acid, ammonium salt
CdCl₂
CrCl₃
AgNO₃
AlCl₃ 6H₂O
Ba (C₂H₃O₂)₂
CoCl₂ 6H₂O
GeO₂
KBr
KI
NaF
RbCl
ZrOCl₂ 8H₂O
Energy substrates
D-Glucose
Sodium pyruvate
Lipids
Linoleic acid
Linolenic Acid
Lipoic acid
Arachidonic acid
Cholesterol
DL-alpha tocopherol-acetate
Myristic acid
Oleic acid
Palmitic acid
Palmitoleic acid
Stearic Acid
Amino Acids
L-Alanine
L-Arginine hydrochloride
L-Asparagine-H₂O
L-Aspartic acid
L-Cysteine-HCl—H₂O
L-Cystine 2HCl
L-Glutamic acid
L-Glutamine
Glycine
L-Histidine-HCl—H₂O
L-Isoleucine
L-Leucine
L-Lysine hydrochloride
L-Methionine
L-Phenylalanine
L-Proline
L-Serine
L-Threonine
L-Tryptophan
L-Tyrosine 2Na 2H₂O
L-Valine
Vitamins
Ascorbic acid
Biotin
B12
Choline chloride
D-Calcium pantothenate
Folic acid
i-Inositol
Niacinamide
Pyridoxine hydrochloride
Riboflavin
Thiamine hydrochloride
Growth Factors and Other Proteins
GABA
Pipecolic acid
bFGF
TGF beta 1
Human Insulin
Human Holo-Transferrin
Human Serum Albumin Glutathione (reduced)
Other Components
Hypoxanthine Na
Phenol red
Putrescine-2HCl
Thymidine
2-mercaptoethanol
Pluronic F-68
Tween 80

Modifications to the medium (mTeSR1) that include the use of animal-sourced proteins (bovine serum albumin (BSA) and Matrigel™) and cloned zebrafish basic fibroblast growth factor (zbFGF) are reported in Ludwig et al. “Feeder-independent culture of human embryonic stem cells,” Nature Methods, volume 3, pages 637-646 (2006). Matrigel™ matrix is a solubilized basement membrane preparation extracted from the Engelbreth-Holm-Swarm (EHS) mouse sarcoma, a tumor rich in extracellular matrix proteins. Matrigel™ a partially defined extracellular matrix (ECM) extract including laminin (a major component), collagen IV, heparan sulfate proteoglycans, entactin/nidogen, and a number of growth factors such as TGF-beta 1, epidermal growth factor, insulin-like growth factor, fibroblast growth factor, tissue plasminogen activator. Braam et al. report that in Matrigel™ or natural and recombinant vitronectin was effective in supporting sustained self-renewal and pluripotency in three independent human embryonic stem cells lines, Stem Cells, 2008, 26(9):2257-65.

Chen et al. report using an E8-based medium, defined conditions can be used for iPS cell derivation and culture, Nat Methods, 2011, 8(5): 424-429. Human ES and iPS cells can be expanded in a medium containing insulin, selenium, transferrin, L-ascorbic acid, bFGF, and TGF-β (or NODAL) in DMEM/F12 with pH adjusted with NaHCO₃. The addition of NODAL (100 ng/ml) or TGF-β1 (2 ng/ml) increased NANOG expression levels and led to consistent long-term culture stability of both human ES and iPS cells. The inclusion of either a ROCK inhibitor (HA100 or Y27632) or blebbistatin improved initial survival and supported cloning, which was further improved by the addition of transferrin and by culture in hypoxic conditions. Multiple matrix proteins, such as laminin, vitronectin and fibronectin, support human ES cell growth.

The term “fluorescence-activated cell sorting” or “FACS” refers to a method of sorting a mixture of cells into two or more areas, typically one cell at a time, based upon the fluorescent characteristics of each cell. It is typically accomplished by applying an electrical charge and separating by movement through an electrostatic field. Fluorescent antibodies with epitopes to cell surface markers can be mixed with cells to mark the cells. Typically, in FACS, a vibrating mechanism causes a stream of cells to break into individual droplets. Just prior to droplet formation, cells in a fluid pass through an area for measuring fluorescence of the cell. An electrical charging mechanism is configured at the point where the stream breaks into droplets. Based on the fluorescence intensity measurement, a respective electrical charge is imposed on the droplet as it breaks from the stream. The charged droplets then move through an electrostatic deflection system that diverts droplets into areas based upon their relative charge. In some systems, the charge is applied directly to the stream, and the droplet breaking off retains charge of the same sign as the stream. In other systems, a charge is provided on a conduit inducing an opposite charge on the droplet.

Variants of proteins disclosed herein can be easily produced by a skilled artisan. One can predict functioning variants with structural similarity using computer modeling. Tests confirming inherent activity can be done using procedures outlined in the literature or in this specification. A skilled artisan would understand that one could produce a large number of operable variants that would be expected to have the desirable properties. Genes are known and members share significant homologies from one species to another. The sequences are not identical as illustrated by the differences between the human and mouse sequences. Some are conserved substitutions. Some are not conserved substitutions. In order to create functioning variants, skilled artisans would not blindly try random combinations, but instead utilize computer programs to make stable substitutions. Skilled artisans would know that certain conserved substations would be desirable. In addition, a skilled artisan would not typically alter evolutionary conserved positions. See Saldano et al. “Evolutionary Conserved Positions Define Protein Conformational Diversity,” PLoS Comput Biol., 2016, 12(3):e1004775.

Guidance in determining which and how many amino acid residues may be substituted, inserted or deleted without abolishing biological activity may be found using computer programs in combination with publicly available databases well known in the art, for example, RaptorX, ESyPred3D, HHpred, Homology Modeling Professional for HyperChem, DNAStar, SPARKS-X, EVfold, Phyre, and Phyre2 software. See Kelley et al. which report the Phyre2 web portal for protein modelling, prediction and analysis, Nat Protoc., 2015, 10(6):845-858. See also Marks et al., Protein structure from sequence variation, Nat Biotechnol, 2012, 30(11):1072-1080; Mackenzie et al., Curr Opin Struct Biol, 2017, 44:161-167; Mackenzie et al., Proc Natl Acad Sci USA., 2016, 113(47), E7438-E7447 and Wei et al., Int. J. Mol. Sci., 2016, 17(12), 2118.

In certain embodiments, this disclosure contemplates using variants of polypeptide sequences disclosed herein having greater than 50%, 60%, 70%, 80%, 90%, 95%, or more identity. “Sequence identity” refers to a measure of relatedness between two or more nucleic acids or proteins, and it is typically given as a percentage with reference to the total comparison length. Identity calculations take into account those amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.) using default parameters. In certain embodiments, sequence “identity” refers to the number of exactly matching residues (expressed as a percentage) in a sequence alignment between two sequences of the alignment. In certain embodiments, percentage identity of an alignment may be calculated using the number of identical positions divided by the greater of the shortest sequence or the number of equivalent positions excluding overhangs wherein internal gaps are counted as an equivalent position. For example, the polypeptides GGGGGG (SEQ ID NO: 6) and GGGGT (SEQ ID NO: 7) have a sequence identity of 4 out of 5 or 80%. For example, the polypeptides GGGPPP (SEQ ID NO: 8) and GGGAPPP (SEQ ID NO: 9) have a sequence identity of 6 out of 7 or 85%.

In certain embodiments, for any contemplated percentage sequence identity, it is also contemplated that the sequence may have the same percentage or more of sequence similarity. Percent “similarity” is used to quantify the extent of similarity, e.g., hydrophobicity, hydrogen bonding potential, electrostatic charge, of amino acids between two sequences of the alignment. This method is similar to determining the identity except that certain amino acids do not have to be identical to have a match. In certain embodiments, sequence similarity may be calculated with well-known computer programs using default parameters. Typically, amino acids are classified as matches if they are among a group with similar properties, e.g., according to the following amino acid groups: Aromatic-F Y W; hydrophobic-A V I L; Charged positive-R K H; Charged negative-D E; Polar-S T N Q.

Generation of Smooth Muscle Cells from Human Pluripotent Stem Cells

Dysfunctional blood vessels are found in cardiovascular ischemic conditions such as myocardial infarction (MI) and peripheral arterial disease (PAD). Often, these diseases involve atherosclerotic occlusion of blood vessels, leading to low blood circulation and eventual tissue necrosis. Introducing more functional blood vessels to the affected area may ameliorate ischemic conditions and improve quality of life.

Vascular smooth muscle cells (VSMCs) are an important component of the vasculature of both blood and lymphatic vessels. High-pressure arteries are covered with multi-layered VSMCs, whereas veins are typically covered by spotty-patterned VSMCs. Lymphatic vessels are sparsely covered with VSMCs. VSMCs contribute to maturation and stabilization of nascent blood vessels, leading to functional vessels.

Induced pluripotent stem cells (iPSCs), e.g., derived from skin or blood cells, possess the capability to grow and to differentiate into various types of somatic cells. Thus, cells differentiated from human iPSCs (hiPSCs) in chemically defined conditions are good sources for a variety of clinical applications. However, chemically defined and clinically compatible differentiation condition of VSMCs from human pluripotent stem cells (hPSCs) including human embryonic stem cells (hESCs) and hiPSCs are needed. Furthermore, due to an absence of a specific surface marker for VSMCs, the selection and purification of VSMCs derived from hiPSCs and hESCs remains challenging.

Human iPSCs were differentiated into VSMCs after GSK3 inhibition and subsequent exposure to specific growth factors, TGF-β1 with PDGF-BB. VSMCs derived from hiPSCs express VSMC specific genes and proteins, such as TAGLN, CNN1, ACTA2, CALD1, SMTN, and MYH11.

To be functional, VSMCs must display a contractile phenotype. The phenotype switching of VSMCs from contractile to synthetic VSMCs causes pathological changes in the vasculature. F-actin formation by polymerization of G-actin indicates mature contractile VSMCs. A co-factor of serum response factor (SRF), myocardin-related transcription factor-A (MRTFA) shuttles between cytoplasm and nucleus, and this shuttling of MRTFA is dependent on its binding to G-actin in the cytoplasm. Upon formation of F-actin polymerized G-actin, MRTFA is released from G-actin and then translocated to the nucleus which leads to activation of SRF.

Matrix metalloproteinases (M1VIPs) play an important role in the remodeling of the tissues, especially vasculature. MMPs degrade extracellular matrix (ECM). In addition to ECM, MMPs degrade peptide growth factors and tyrosine kinase receptors, cell-adhesion molecules, cytokines and chemokines, as well as other MMPs. MMP2 and MMP9 release TGFβ from an inactive extracellular complex, consisting of TGFβ, TGFβ latency-associated protein (which is the pro-domain of TGFβ), and latent TGFβ-binding protein. MMP2- and MMP9-mediated TGFβ activation leads to angiogenesis.

The primary cadherin in SMCs, cadherin 2 (CDH2) has been expressed and identified in the rat vasculature. Upregulation of CDH2 occurs upon differentiation of SMC from human mesenchymal stem cells (hMSCs). CDH2 is important for maturation of endothelial sprouts by interaction with pericytes and for neovascularization. Recruitment of β-catenin to transmembrane CDH2 is regulated by actin polymerization, and this process is important for SMC contraction. The cleavage of the extracellular domain of CDH2 by MMP9 and MMP12 triggers β-catenin signaling and cyclin D1 expression in VSMCs, leading to increased proliferation of VSMCs.

VSMCs were generated from hPSCs in chemically defined condition. hiPSCs were plated on collagen-coated dishes in animal feeder cell-free condition. Only GSK-inhibitor CHIR-99021 and bFGF were used for mesoderm induction and only three growth factors, TGF-β1, PDGF-BB, and EGF for differentiation of VSMCs from hiPSCs. To generate VSMCs efficiently, the reseeding cells on Day 10 after detaching by Accutase™ and filtration was performed. Accutase™ consists of a mixture of proteolytic and collagenolytic enzymes and Na₄EDTA which is used as a mild detaching agent without cutting-off the extracellular domain of transmembrane proteins at the cell surface.

Moreover, two different VSMC-differentiation media were used with different concentration of TGF-β1 and PDGF-BB, but with the same concentration of EGF. Contractile VSMCs were generated in VSMC-differentiation medium II with increased concentration of TGF-β1 (from 2.5 ng/ml to 5.0 ng/ml) and decreased concentration of PDGF-BB (from 5.0 ng/ml to 2.5 ng/ml) from day 25. Furthermore, CDH2 was discovered to be a selection marker on the surface of hiPSC-VSMCs. The selection of differentiated hiPSC-VSMCs by CDH2 as a selection surface marker leads to efficiently differentiated and enriched contractile VSMCs from hiPSCs. These contractile hiPSC-VSMCs were generated by a simple, chemically defined, and animal product free conditions and show therapeutic potential in remodeling and regeneration of vasculature.

### Methods of Use

In certain embodiments, methods disclosed herein comprise contacting pluripotent stem cells with a mesoderm induction growth media, followed by replicating the cells in a serum-free vascular smooth muscle cell growth medium in the presence of collagen, and purifying replicated cells that express cadherin-2. In certain embodiments, the purified cells are used to treat or prevent cardiovascular diseases or conditions.

In certain embodiments, this disclosure relates to methods of producing vascular smooth muscle like cells in a contractile phenotype comprising transforming pluripotent stem cells into cells that express smoothelin and smooth muscle myosin heavy chain and purifying cells that express cadherin-2 providing a purified composition of cadherin-2 expressing vascular smooth muscle like cells in a contractile phenotype. In certain embodiments, the method further comprises replicating cadherin-2 expressing vascular smooth muscle like cells.

In certain embodiments, this disclosure relates to a serum-free method making vascular smooth muscle like cells comprising, a) contacting pluripotent stem cells with a mesoderm induction growth medium for a day or more, wherein the mesoderm induction growth medium comprises: 1) rho-associated protein kinase inhibitor, 2) glycogen synthase kinase-3 inhibitor, and 3) basic fibroblast growth factor; under conditions such that the pluripotent stem cells form induced mesodermal-like cells; contacting the induced mesodermal-like cells with a first vascular smooth muscle cell growth medium for a day or more, wherein the first vascular smooth muscle cell growth medium comprises: 1) transforming growth factor-beta, 2) epidermal growth factor, and 3) platelet-derived growth factor; under conditions such that the mesodermal-like cells form induced vascular smooth muscle like cells; c) contacting the induced vascular smooth muscle like cells with a protease and/or collagenase under conditions such that induced vascular smooth muscle like cells detach from each other providing detached induced vascular smooth muscle like cells; d) replicating the detached induced vascular smooth muscle like cells by exposure to collagen and the first vascular smooth muscle cell growth medium for a day or more providing replicated vascular smooth muscle like cells; e) contacting replicated vascular smooth muscle like cells with a second vascular smooth muscle cell growth medium for a day or more, wherein the second vascular smooth muscle cell growth medium comprises: 1) transforming growth factor-beta, 2) epidermal growth factor, and 3) platelet-derived growth factor; under conditions such that the replicated vascular smooth muscle like cells form a second batch of induced vascular smooth muscle like cells; and f) purifying the second batch of induced vascular smooth muscle like cells by selecting cells that express cadherin-2, providing purified cadherin-2 expressing induced vascular smooth muscle like cells.

In certain embodiments, the concentration of transforming growth factor-beta in the second vascular smooth muscle cell growth medium is increased compared to the concentration of transforming growth factor-beta in the first vascular smooth muscle cell growth media.

In certain embodiments, the concentration of platelet-derived growth factor in the second vascular smooth muscle cell growth medium is decreased compared to the concentration of platelet-derived growth factor in the first vascular smooth muscle cell growth media.

In certain embodiments, the rho-associated protein kinase inhibitor is trans-4-[(1R)-1-Aminoethyl]-N-4-pyridinylcyclohexanecarboxamide (Y-27632) or salt thereof.

In certain embodiments, the glycogen synthase kinase-3 inhibitor is 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR-99021) or salt thereof.

In certain embodiments, the pluripotent stem cells are embryonic stem (ES) cells or induced pluripotent stem (iPS) cells.

In certain embodiments, said contacting pluripotent stem cells with a mesoderm induction growth medium for a day or more comprises contacting for four days.

In certain embodiments, said contacting pluripotent stem cells with a mesoderm induction growth medium for a day or more is for not more than five days.

In certain embodiments, said contacting the induced mesodermal-like cells with a first vascular smooth muscle cell growth medium for a day or more comprises contacting for twenty days.

In certain embodiments, said contacting the induced mesodermal-like cells with a first vascular smooth muscle cell growth medium for a day or more, is for not more than 21 days.

In certain embodiments, said replicating the detached induced vascular smooth muscle like cells by exposure to collagen and the first vascular smooth muscle cell growth medium for a day or more comprises replicating for fifteen days.

In certain embodiments, said replicating the detached induced vascular smooth muscle like cells by exposure to collagen and the first vascular smooth muscle cell growth medium for a day or more is for not more than 16 days.

In certain embodiments, said contacting replicated vascular smooth muscle like cells with a second vascular smooth muscle cell growth medium for a day or more comprises contacting for twenty-five days or more.

In certain embodiments, said contacting replicated vascular smooth muscle like cells with a second vascular smooth muscle cell growth medium for a day or more is not more than 26 days.

In certain embodiments, the methods disclosed herein further comprises the step of replicating the purified cadherin-2 (CDH2) expressing induced vascular smooth muscle like cells by exposing the purified cadherin-2 (CDH2) expressing induced vascular smooth muscle like cells to collagen and the second vascular smooth muscle cell growth medium for a day or more.

In certain embodiments, said selecting cells that express cadherin-2 (CDH2) comprises contacting the cells with an anti-cadherin-2 antibody, marking the cell surface expressing cadherin-2 (CDH2) with the antibody conjugated with fluorescence, and selecting cells by fluorescence-activated cell sorting.

In certain embodiments, this disclosure relates to compositions and growth media comprising cells made by methods disclosed herein.

In certain embodiments, this disclosure relates to methods of treating or preventing a cardiovascular disease or condition comprising administering an effective amount of cells made by methods disclosed herein to a subject in need thereof. In certain embodiments, the pluripotent stem cells are induced pluripotent stem cells derived from the subject.

In certain embodiments, the subject is diagnosed with or at risk of damaged and narrowed arteries, aneurysm, angina, arrhythmias, enlarged left heart, transient ischemic attack, stroke, dementia, kidney scarring, kidney failure, myocardial infarction, vascular inflammation, plaque formation, atherosclerosis, restenosis, pulmonary hypertension, ocular hypertension, retinopathy, choroidopathy, optic neuropathy, glaucoma, and blindness.

In certain embodiments, vascular smooth muscle like cells contract in response to vasoactive agents such as carbachol or KCl.

In certain embodiments, vascular smooth muscle like cells are mixed with or administered in combination with and endothelial cells or endothelial like cells resulting in improved blood flow restoration compared with the administration of endothelial cells or endothelial like cells alone.

In certain embodiments, vascular smooth muscle like cells are mixed with or administered in combination with and endothelial cells or endothelial like cells creating capillary-like tubes.

In certain embodiments, this disclosure relates to using CDH2 as a biomarker for isolating vascular smooth muscle cells generated from human induced pluripotent stem cells. In certain embodiments, this disclosure contemplates mixing a sample of cells suspected of containing smooth muscle cells and a specific binding agent for CDH2 under conditions such that the specific binding agent for CDH2 (e.g., CDH2 antibody) binds CDH2, and measuring and/or detecting the specific binding.

In certain embodiments, this disclosure relates to engineered blood vessels to treat ischemic conditions. In certain embodiments, tubes of mixtures of endothelial cells and vascular smooth muscle like-cells described herein are produced in vitro and thereafter implanted into a subject in need thereof. In certain embodiments, this disclosure contemplates administering vascular smooth muscle like-cells described herein, optionally in combination with endothelial cells, in vivo whereby the cells circulate and are effective at cites of ischemic injury. In certain embodiments, this disclosure contemplates administering vascular smooth muscle like-cells described herein, optionally in combination with endothelial cells, locally to an area of ischemic injury, e.g., by direct injection to a vein, arteries, the heart or area surrounding or near the heart.

In certain embodiments, this disclosure relates to methods using hiPSC-VSMCs applied directly to the ischemic area. It is contemplated that VSMCs localize to adjacent nascent blood vessels and stabilize the blood vessel formation. In addition to the role of VSMCs in the stabilization of blood vessel formation, VSMCs express direct angiogenic factors, such as VEGF and bFGF, when cells are incubated with indirect angiogenic cytokines, such as PDGF-BB and TGF-β1. In hypoxic condition, VSMCs express VEGF. Expression and secretion of MMPs are other beneficial factors in angiogenic vascular remodeling.

In certain embodiments, this disclosure relates to methods of generation of blood vessels ex-vivo and implantation into a subject. In certain embodiments, the blood vessels are generated by culturing vascular smooth muscle like-cells described herein, optionally in combination with endothelial cells in the form of tubes or sheets or deforming the sheets into cylinder like structures. In certain embodiments this disclosure contemplates taking the sheets and applying them locally to the ischemic vasculature.

In certain embodiment on contemplates extracting in vivo vasculature, e.g., vein or an artery, and contacting the vasculature with vascular smooth muscle like-cells described herein, optionally in combination with endothelial cells providing exposed vasculature, and then implanting exposed vasculature into a subject.

In certain embodiments, this disclosure relates to methods of drug screening using vascular smooth muscle like-cells described herein. In certain embodiments, one contacts vascular smooth muscle like-cells described herein, optionally in combination with endothelial cells, and a test agent. In certain embodiments, vascular smooth muscle like-cells described herein are derived from induced pluripotent stem cell derived a subject with a vascular defect and/or genetic profile indicative of a subject at risk of an undesirable cardiovascular conditions. One observes whether the test agent induces a phenotypic change, e.g., when compared to a control agent. For example, one can use vascular smooth muscle like-cells described herein to determine an increase or decrease of contractility in the presence of the test agent.

There are disease models wherein genomic mutation affect VSMCs that are associated with a patient's disease including the congenital heart defect associated with supravalvular aortic stenosis (SVAS), Williams-Beuren syndrome (WBS), Hutchison Gilford Progeria (HGP) syndrome, and Marfan syndrome. Thus, vascular smooth muscle like-cells described herein can be produced from induced pluripotent stem cells derived from a subject having one or more of these diseases or conditions. Thereafter, the vascular smooth muscle like-cells made by methods disclosed herein derived from a subject associated with one of these conditions can be use in a drug screening library to identify therapeutic agents. See Granata et al. “An iPSC-derived vascular model of Marfan syndrome identifies key mediators of smooth muscle cell death,” Nat Genet., 49, 97-109 (2017).

Process for Generating hiPSC-VSMCs:

The human induced pluripotent stem cells (hiPSCs) were cultured in mTeSRTM™ (STEMCELL Technologies, Cat #85850) on 5% Matrigel™ (Corning, Cat #354234) at 37° C. with 5% CO₂. For the VSMC differentiation, three different media, mesoderm-induction medium, VSMC-differentiation medium I, and VSMC-differentiation medium II were used. These media contain small molecules and growth factors in basal medium containing 20% Knockout™ Serum Replacement (KO-SR, Invitrogen, Cat #10828-028). In Basal medium DMEM/F12 (Invitrogen, Cat #11330057) was supplemented with anti-microbial agents (Gibco, Cat #15240-112), MEM NEAA (Gibco, Cat #11140076), and GlutaMax™ (Gibco, Cat #35050079). hiPSCs were plated on 0.01% Collagen (STEMCELL Technologies, Cat #4902)-coated plates after dissociation using Dispase (Gibco, Cat #17105-041).

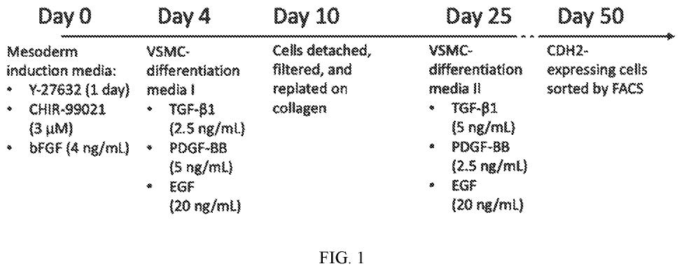

On day 0, hiPSCs were cultured in Basal medium containing 20% Knockout™ Serum Replacement with Y-27632 (STEMCELL Technologies, Cat #72304), 3 μM CHIR-99021 (GSK inhibitor) (Selleckchem, Cat #S1263), and bFGF (4 ng/ml) to induce the mesodermal lineage. The ROCK inhibitor Y-27632 was used only on day 0. The mesoderm induction medium was renewed every day. (See FIG. 1).

On day 4, mesoderm induction medium was replaced with VSMC-differentiation medium I, containing TGF-β1 (2.5 ng/ml, PeproTech, Cat #100-21C), PDGF-BB (5 ng/ml, PeproTech, Cat #100-14B), EGF (20 ng/ml, R&D System, Cat #236-EG-200), and 20% Knockout™ Serum Replacement.

On day 10, cells were replated on the collagen-coated plate after detaching by Accutase (eBioscience, Cat #00-4555-56), followed by filtration through Cell Strainer (70 μm Nylon Mesh, Fisher Scientific, Cat #22363548). VSMC-differentiation medium I was renewed every day.

From day 25, cells were cultured in VSMC-differentiation medium II, containing TGF-β1 (5 ng/ml), PDGF-BB (2.5 ng/ml), EGF (20 ng/ml), and 20% Knockout™ Serum Replacement VSMC-differentiation medium II was renewed every day.

On day 50, CDH2-expressing cells were sorted by FACS after labeling cells with PE-conjugated anti CDH2 antibody (eBioscience, Cat #12-3259-42) at 4° C. After selection, CDH2-positive cells were further cultured on the collagen-coated plate in VSMC-differentiation medium II. VSMC-differentiation medium II was renewed every day. Cells were split every 3-4 days when cells became confluent.

### Evaluation of Cellular Changes

Maintenance of contractile phenotype of VMSCs using growth factors is reported. The differentiation of VSMCs from human ESC and iPSCs is not simple. VSMCs were generated from human PSCs including ESCs and iPSCs. Human PSCs were maintained in feeder cell-free condition. VSMCs were differentiated in chemically defined condition without animal serum supplementation. Moreover, CDH2-positive selection leads to enriched contractile VSMCs.

Cheung et al. (Nature Protocols, 2014) report around 80% of their differentiated VSMCs with PDGF-BB (10 ng/ml) and TGF-β1 (2 ng/ml) are CNN1 and MYH11 double positive. However, a low number of cells are CNN1 and TAGLN positive even though ACTA2, CNN1 and TAGLN are early SMC markers. Patsch et al. (Nature Cell Biology, 2015) differentiated VSMCs and report counted ACTA2 (48%), Myosin IIB (96.99%), and TAGLN (100%) positive cells. Myosin IIB (MYH10) is not a VSMC marker. A high number of cells were CD140b positive. Fibroblast express CD140b as well.

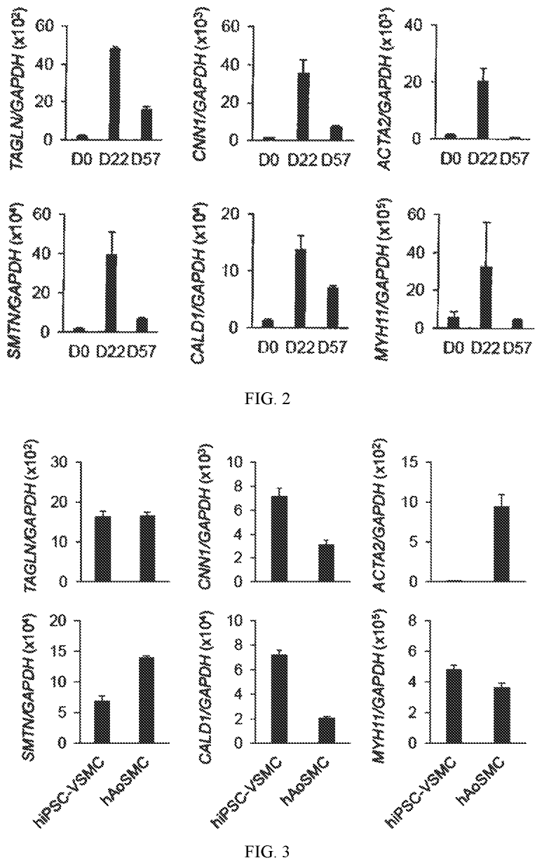

ACTA2, CNN1 and TAGLN are early smooth muscle cell markers. The mRNA expression level of VSMC specific genes such as TAGLN, CNN1, ACTA2, SMTN, CALD1, and MYH11 was significantly increased on day 22 (FIG. 2). However, this mRNA expression level of VSMC specific genes in hiPSC-VSMCs was similar or higher than that in human aortic smooth muscle cells (hAoSMCs), except ACTA2 and SMTN (FIG. 3).

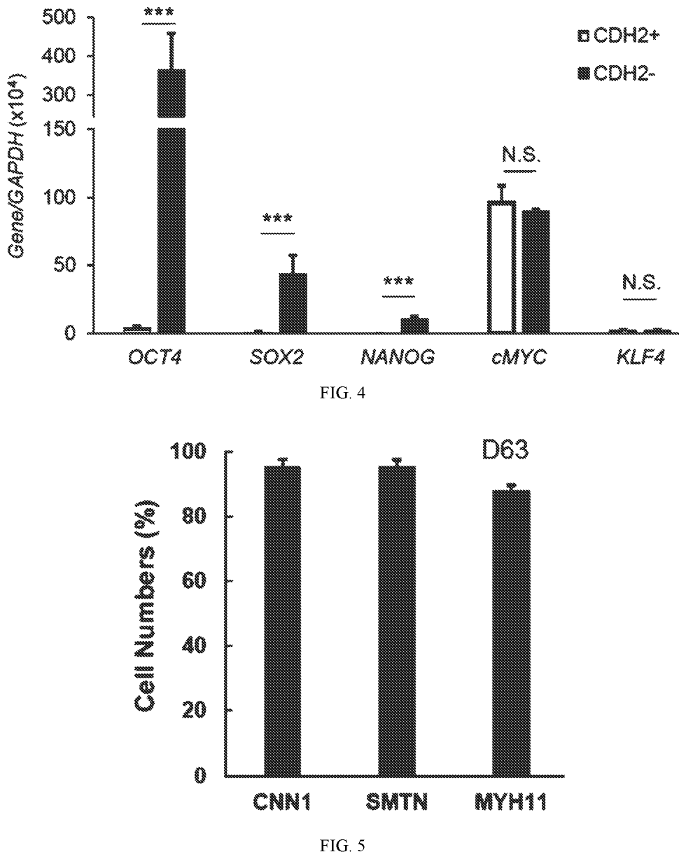

The expression of pluripotency-related genes such as OCT4, SOX2, NANOG, and KLF4 was significantly higher in CDH2-negative fraction, but the expression level of cMYC was similar in CDH2-negative and -positive (FIG. 4). The formation of F-actin and the expression of MRTFA in hiPSC-VSMCs were observed by immunofluorescence microscopy on day 49. Furthermore, the localization of MRTFA in the nucleus indicates the promotion of contractile gene expression. The expression of contractile VSMC proteins such as ACTA2, CNN1, SMTN and MYH11 in CDH2-positive hiPSC-VSMCs is observed on day 58. Flow cytometry analysis after intracellular staining by BD LSRII shows the expression of CNN1 (95.23±2.15), SMTN (95.37±1.96), and MYH11 (87.87±0.94) in hiPSC-VSMCs on day 63 (FIG. 5). Long isoform of SMTN (SMTN-B) is the late expressing vascular smooth muscle cell marker. It is challenging to detect in primary cells. The expression of SMTN was dramatically decreased when culturing the primary cells. However, VSMCs derived from human PSCs (hPSC-VSMCs) express SMTN. The expression level of the long isoform of SMTN (SMTN-B) was confirmed in the differentiated hPSC-VSMCs.

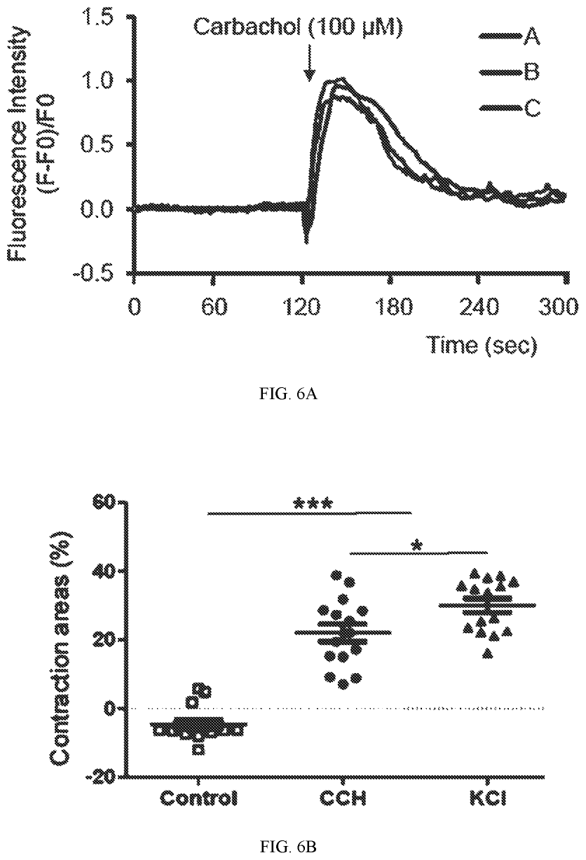

A dramatic increase of Fluo4 fluorescence by carbachol indicates the increased intracellular calcium flux in hiPSC-VSMCs (FIG. 6A). Additionally, significantly increased contractility was observed in hiPSC-VSMCs treated with carbachol and KCl. A significant difference also between cells treated with carbachol and potassium chloride was observed (FIG. 6B).

The Therapeutic Potential of hiPSC-VSMCs in the Context of Angiogenesis

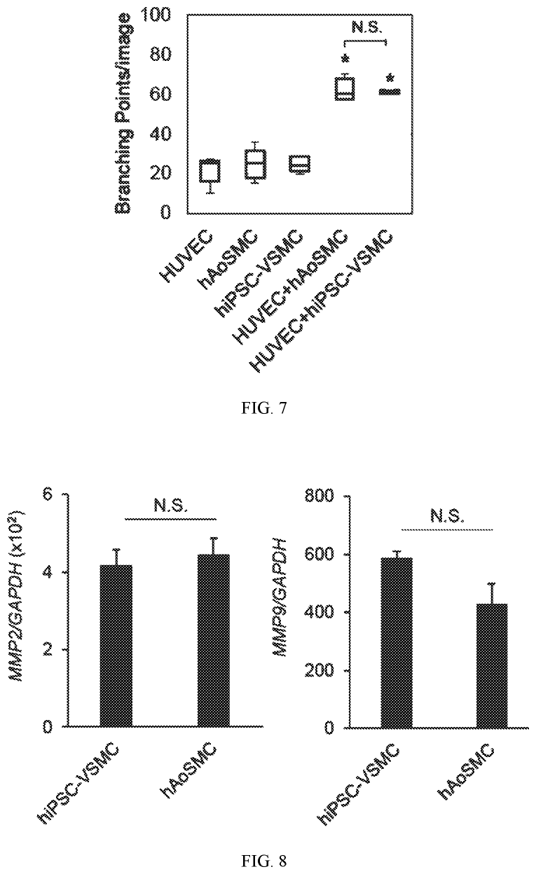

Pre-stained cells were incubated on the Matrigel™ with reduced growth factors in VSMC-differentiation medium I with VEGFA (10 ng/ml). HUVECs were pre-stained with DiI (red), and hAoSMCs and hiPSC-VSMCs were pre-stained with DiO (green). Branching points were quantified from 5 different images per group. Tube formation with significant branching points was observed when hiPSC-VSMCs were co-cultured with HUVECs. No significant difference in branching points was observed between co-culture of hiPSC-VSMCs and hAoSMCs with HUVEC (FIG. 7).

MMPs play a critical role in the remodeling of the vasculature. The expression level of MMP2 and MMP9 in hiPSC-VSMCs was determined. hiPSC-VSMCs express similar level of mRNA of WP2 and WP9 compared to that of hAoSMCs (FIG. 8).

1. A method making vascular smooth muscle like cells comprising,
a) contacting pluripotent stem cells with a mesoderm induction growth medium for a day or more, wherein the mesoderm induction growth medium comprises:
1) rho-associated protein kinase inhibitor,
2) glycogen synthase kinase-3 inhibitor, and
3) basic fibroblast growth factor;
under conditions such that the pluripotent stem cells form induced mesodermal-like cells;
b) contacting the induced mesodermal-like cells with a first vascular smooth muscle cell growth medium for a day or more, wherein the first vascular smooth muscle cell growth medium comprises:
1) transforming growth factor-beta,
2) epidermal growth factor, and
3) platelet-derived growth factor;
under conditions such that the mesodermal-like cells form induced vascular smooth muscle like cells;
c) contacting the induced vascular smooth muscle like cells with a protease or collagenase under conditions such that induced vascular smooth muscle like cells detach from each other providing detached induced vascular smooth muscle like cells;
d) replicating the detached induced vascular smooth muscle like cells by exposure to collagen and the first vascular smooth muscle cell growth medium for a day or more providing replicated vascular smooth muscle like cells;
e) contacting replicated vascular smooth muscle like cells with a second vascular smooth muscle cell growth medium for a day or more, wherein the second vascular smooth muscle cell growth medium comprises:
1) transforming growth factor-beta,
2) epidermal growth factor, and
3) platelet-derived growth factor;
under conditions such that the replicated vascular smooth muscle like cells form a second batch of induced vascular smooth muscle like cells; and
f) purifying the second batch of induced vascular smooth muscle like cells by selecting cells that express cadherin-2, providing purified cadherin-2 expressing induced vascular smooth muscle like cells.

2. The method of claim 1, wherein the concentration of transforming growth factor-beta in the second vascular smooth muscle cell growth medium is increased compared to the concentration of transforming growth factor-beta in the first vascular smooth muscle cell growth media.

3. The method of claim 2, wherein the concentration of platelet-derived growth factor in the second vascular smooth muscle cell growth medium is decreased compared to the concentration of platelet-derived growth factor in the first vascular smooth muscle cell growth media.

4. The method of claim 1, wherein the rho-associated protein kinase inhibitor is trans-4-[(1R)-1-aminoethyl]-N-4-pyridinylcyclohexanecarboxamide (Y-27632) or salt thereof.

5. The method of claim 1, wherein the glycogen synthase kinase-3 inhibitor is 6-[[2-[[4-(2,4-dichlorophenyl)-5-(5-methyl-1H-imidazol-2-yl)-2-pyrimidinyl]amino]ethyl]amino]-3-pyridine-carbonitrile (CHIR-99021) or salt thereof.

6. The method of claim 1, wherein the pluripotent stem cells are embryonic stem (ES) cells or induced pluripotent stem (iPS) cells.

7. The method of claim 1, wherein contacting pluripotent stem cells with a mesoderm induction growth medium for a day or more is for four days.

8. The method of claim 1, wherein contacting pluripotent stem cells with a mesoderm induction growth medium for a day or more is for not more than five days.

9. The method of claim 1, wherein contacting the induced mesodermal-like cells with a first vascular smooth muscle cell growth medium for a day or more, is for twenty days.

10. The method of claim 1, wherein contacting the induced mesodermal-like cells with a first vascular smooth muscle cell growth medium for a day or more, is for not more than 21 days.

11. The method of claim 1, wherein replicating the detached induced vascular smooth muscle like cells by exposure to collagen and the first vascular smooth muscle cell growth medium for a day or more is for fifteen days.

12. The method of claim 1, wherein replicating the detached induced vascular smooth muscle like cells by exposure to collagen and the first vascular smooth muscle cell growth medium for a day or more is for not more than 16 days.

13. The method of claim 1, wherein contacting replicated vascular smooth muscle like cells with a second vascular smooth muscle cell growth medium for a day or more is twenty-five days or more.

14. The method of claim 1, wherein contacting replicated vascular smooth muscle like cells with a second vascular smooth muscle cell growth medium for a day or more is not more than 26 days.

15. The method of claim 1, wherein the method further comprises the step of replicating the purified cadherin-2 expressing induced vascular smooth muscle like cells by exposing the purified cadherin-2 expressing induced vascular smooth muscle like cells to collagen and the second vascular smooth muscle cell growth medium for a day or more.

16. A method or producing vascular smooth muscle like cells comprising transforming pluripotent stem cells into cells that express vascular smoothelin and vascular smooth muscle myosin heavy chain and purifying cells that express cadherin-2 providing vascular smooth muscle like cells.

17. A composition comprising cells made by the method of claim 16.

18. A method of treating or preventing a cardiovascular disease or condition comprising administering an effective amount of cells made by the method described in claim 1 to a subject in need thereof.

19. The method of claim 18, wherein the pluripotent cells are induced pluripotent cells derived from the subject.
